(19) United States
(12) Patent Application Publication
(10) Pub. No.: US 2017/0097315 A1
(43) Pub. Date: Apr. 6, 2017

(54) SUBSTRATE FOR STRETCHABLE ELECTRONIC DEVICE, METHOD OF MANUFACTURING THE SUBSTRATE, AND ELECTRONIC DEVICE HAVING THE SUBSTRATE

(71) Applicant: RESEARCH & BUSINESS FOUNDATION SUNGKYUNKWAN UNIVERSITY, Suwon-si (KR)

(72) Inventors: NAE-EUNG LEE, Seoul (KR); HAN-BYEOL LEE, Incheon (KR); CHAN-WOOL BAE, Suwon-si (KR)

(73) Assignee: RESEARCH & BUSINESS FOUNDATION SUNGKYUNKWAN UNIVER SITY, Suwon-si (KR)

(21) Appl. No.: 15/258,329

(22) Filed: Sep. 7, 2016

(30) Foreign Application Priority Data
Sep. 7, 2015 (KR) 10-2015-0126304

(51) Int. Cl. G01N 27/22; G01N 33/00; H05K 1/09

(52) U.S. Cl. CPC G01N 27/221; H05K 1/09; G01N 33/0009; H05K 2201/0367; H05K 2201/0323; H05K 2201/0329; G01N 2027/222

(57) ABSTRACT

There is provided a substrate for a stretchable device, the substrate having a mogul pattern formed thereon, wherein the mogul pattern has a plurality of bumps protruding from a virtual reference plane, and a continuous valley formed between the bumps, wherein the valley surrounds the bumps, and the bumps are regularly or irregularly arranged and have substantially the same size and shape, wherein a combination of the bumps and the valley has a continuous curved surface.

### CROSS-REFERENCE TO RELATED APPLICATION

This application claims the benefit of Korea Patent Application No. 10-2015-0126304 filed on, Sep. 7, 2015, the entire content of which is incorporated herein by reference for all purposes as if fully set forth herein.

### BACKGROUND

Field of the Present Disclosure

The present disclosure relates to a substrate for a stretchable electronic device, a method of manufacturing the substrate, and an electronic device having the substrate.

Discussion of the Related Art

A stretchable electronic device may have a wide application and, thus, have received much interest. A method for manufacturing the stretchable electronic device may include a direct use of a stretchable material or a minimization of a strain resulting from absorption of a stress thereto using a structural design.

As for the direct use of a stretchable material, the components of the stretchable electronic device may be made of a stretchable material. However, this approach may have shortcomings that the stretchable material applied thereto is limited, and a metal or ceramic with superior stability and electrical property to the polymer material could not be applied thereto.

A typical example of using the minimization of a strain resulting from absorption of a stress thereto using a structural design may be that a thin-film structure is formed on a stretchable substrate previously strained by a predetermined deformation amount, and the substrate is recovered to form a corrugated thin-film structure. However, as for the stretchable electronic device using this approach, the thin-film structure may have cracks therein during the production thereof, and the integration level of the device may be lowered, and the production process thereof may be complicated. Further, a peeling between the substrate and thin-film structure may easily occur. Further, the stretching direction may be limited.

### SUMMARY

Thus, the present disclosure provides a substrate for a stretchable device having a mogul pattern formed thereon, to minimize a deformation of the thin-film structure formed thereon.

Further, the present disclosure provides a method for manufacturing the substrate for the stretchable device.

Furthermore, the present disclosure provides an electronic device having the substrate for the stretchable device.

In one aspect, there is provided a substrate for a stretchable device, the substrate having a mogul pattern formed thereon, wherein the mogul pattern has a plurality of bumps protruding from a virtual reference plane, and a continuous valley formed between the bumps, wherein the valley surrounds the bumps, and the bumps are regularly or irregularly arranged and have substantially the same size and shape, wherein a combination of the bumps and the valley has a continuous curved surface.

In one aspect, there is provided a substrate for a stretchable device, the substrate having a mogul pattern formed thereon, wherein the mogul pattern has a plurality of depressions depressed from a virtual reference plane, and a continuous ridge formed between the depressions, wherein the ridge surrounds the depressions, and the depressions are regularly arranged and have substantially the same size and shape, wherein a combination of the depressions and the ridge has a continuous curved surface.

In one implementation, a cross-section of the mogul pattern perpendicular to the virtual reference plane has peaks and valleys, wherein the peaks and valleys are repeatedly alternated and a combination of the peaks and valleys has a continuous curved line.

In one implementation, a ratio between a pitch between neighboring peaks and a height from each valley to each peak is in a range of about 1:0.5 to 1:1.5.

In one aspect, there is provided a method for manufacturing a substrate for a stretchable device, the method comprising: (a) forming a first photoresist pattern on a first plate by a photolithography process using a first mask, wherein the first mask has a plurality of first light-transmitting regions spacedly arranged regularly and a first light-blocking region surrounding the first light-transmitting regions, wherein each light-transmitting region has a circular or polygonal shape; (b) reflowing the first photoresist pattern; (c) applying and curing a first curable polymer material on an exposed face of the first plate and on the reflowed first photoresist pattern, and acquiring a second plate having a reverse pattern formed thereon, wherein the reverse pattern is shape-reverse to the reflowed first photoresist pattern, wherein the reverse pattern is made of the first curable polymer material; (d) forming a second photoresist pattern on the reverse pattern by a photolithography process using a second mask, wherein a non-removed portion of the second photoresist pattern overlaps a protrusion of the reverse pattern, and a removed portion of the second photoresist pattern overlaps a non-protrusion of the reverse pattern, wherein the second mask has second light-blocking regions corresponding to the first light-transmitting regions respectively, and a second light-transmitting region corresponding to the first light-blocking region; (e) reflowing the second photoresist pattern; and (f) applying and curing a second curable polymer material on the reflowed second photoresist pattern and a non-protrusion of the reverse pattern, and acquiring the substrate having a mogul pattern formed therein, wherein the mogul pattern is shape-reverse to a combination of the reflowed second photoresist pattern and the non-protrusion of the reverse pattern, wherein the mogul pattern is made of the second curable polymer material.

In one implementation, the operation (a) includes forming a first photoresist film on the first plate and patterning the first photoresist film using the first mask, wherein the first photoresist film has a thickness of about 10 to 40 μm.

In one implementation, the operation (b) includes forming a second photoresist film on the reverse pattern and patterning the second photoresist film using the second mask, wherein the second photoresist film has a thickness of about 20 to 50 μm.

In one implementation, each of the first light-transmitting regions in the first mask has a circular shape with a diameter of about 10 to 100 μm.

In one implementation, the combination of the reflowed second photoresist pattern and the non-protrusion of the reverse pattern has a continuous curved surface, and, thus, the mogul pattern has a continuous curved surface.

In one implementation, the operation (c) includes: forming a release film on the reflowed first photoresist pattern; applying the first curable polymer material on the release film; pressuring the first curable polymer material using a glass substrate with improved adhesion to the first curable polymer material; curing the first curable polymer material; and removing the cured first curable polymer material from the reflowed first photoresist pattern using the release film.

In one implementation, the method of claim further comprises applying and curing a third curable polymer material on the mogul pattern and acquiring a master mold having a reverse mogul pattern formed thereon, wherein the reverse mogul pattern is made of the third curable polymer material, and the reverse mogul pattern is shape-reverse to the mogul pattern; and applying and curing the second curable polymer material on the reverse mogul pattern and acquiring the substrate having the mogul pattern formed thereon, wherein the mogul pattern is made of the second curable polymer material.

In one implementation, the third curable polymer material includes polyurethane acrylate.

In one aspect, there is provided an electronic device comprising: a substrate having a mogul pattern formed thereon, wherein the mogul pattern has a plurality of bumps protruding from a virtual reference plane, and a continuous valley formed between the bumps, wherein the valley surrounds the bumps, and the peaks are regularly or irregularly arranged and have substantially the same size and shape, wherein a combination of the bumps and the valley has a continuous curved surface; and an electrical conductive thin-film structure formed on the mogul pattern.

In one implementation, the electrical conductive thin-film structure is made of a metal, a conducive polymer, and/or a conductive oxide.

In one implementation, the electronic device is a sensing device, wherein the electrical conductive thin-film structure includes first electrode and second electrodes spaced from each other, wherein the electronic device is disposed on the mogul pattern, and wherein the electronic device further comprises a sensing material to sense a target material, wherein the sensing material electrically contacts the first electrode and the second electrode.

In one implementation, the sensing material includes a reduced graphene oxide.

In accordance with the present disclosure, the substrate for a stretchable device has the mogul pattern having only the continuous curved face, and, thus, the deformation of the thin-film structure formed on the mogul pattern may be minimized during the tensile force application thereto. As a result, when the substrate is applied to the stretchable electronic device, a stretchable ability of the device may improve in a wide range even when the device has the thin-film structure made of a metal or ceramic.

### BRIEF DESCRIPTION OF THE DRAWINGS

The accompanying drawings, which are incorporated in and form a part of this specification and in which like numerals depict like elements, illustrate embodiments of the present disclosure and, together with the description, serve to explain the principles of the disclosure.

FIG. 1 shows a flow chart of a method for manufacturing a substrate for a stretchable device in accordance with one embodiment of the present disclosure.

FIG. 2 and FIG. 3 shows top views of first and second masks as employed in the process in FIG. 1 respectively.

FIG. 4A to FIG. 4K shows cross-sectional views of stages of the manufacturing process for the substrate for the stretchable device in FIG. 1.

FIG. 5A shows an image of a substrate for a stretchable device in accordance with one embodiment of the present disclosure. FIG. 5B shows a cross-sectional view taken a line A-A′ in FIG. 5A.

FIG. 6 shows a graph indicating resistance variations of gold thin-film patterns based on tensile strains of two substrates, wherein one substrate is a conventional flat substrate and the other is a substrate for a stretchable device, having a mogul pattern formed thereon, in accordance with one embodiment of the present disclosure, wherein the gold thin-film patterns are formed on the two substrates respectively, wherein each of the gold thin-film patterns has an initial length 1 cm and thickness 70 nm.

FIG. 7 shows a graph indicating resistance variations of PEDOT:PSS thin-film patterns based on tensile strains of two substrates, wherein one substrate is a conventional flat substrate and the other is a substrate for a stretchable device, having a mogul pattern formed thereon, in accordance with one embodiment of the present disclosure, wherein the PEDOT:PSS thin-film patterns are formed on the two substrates respectively, wherein each of the PEDOT:PSS thin-film patterns has an initial length 1 cm and thickness 160 nm.

FIG. 8 shows a graph indicating resistance variations of ITO thin-film patterns based on tensile strains of two substrates, wherein one substrate is a conventional flat substrate and the other is a substrate for a stretchable device, having a mogul pattern formed thereon, in accordance with one embodiment of the present disclosure, wherein the ITO thin-film patterns are formed on the two substrates respectively, wherein each of the ITO thin-film patterns has an initial length 1 cm and thickness 160 nm.

FIG. 9 shows a perspective view of a gas sensing device in accordance with one embodiment of the present disclosure.

FIG. 10 shows a graph indicating a variation in the electrical conductance of the gas sensing device based on NO₂ concentrations. As for the gas

### DETAILED DESCRIPTIONS

Examples of various embodiments are illustrated in the accompanying drawings and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

Example embodiments will be described in more detail with reference to the accompanying drawings. The present disclosure, however, may be embodied in various different forms, and should not be construed as being limited to only the illustrated embodiments herein. Rather, these embodiments are provided as examples so that this disclosure will be thorough and complete, and will fully convey the aspects and features of the present disclosure to those skilled in the art.

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

It will be understood that when an element or layer is referred to as being “connected to”, or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, s, operations, elements, and/or components, but do not preclude the presence or addition of one or more other features, integers, s, operations, elements, components, and/or portions thereof. As used herein, the term “and/or” includes any and all combinations of one or more of the associated listed items. Expression such as “at least one of” when preceding a list of elements may modify the entire list of elements and may not modify the individual elements of the list.

Spatially relative terms, such as “beneath,” “below,” “lower,” “under,” “above,” “upper,” and the like, may be used herein for ease of explanation to describe one element or feature's relationship to another element s or feature s as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented for example, rotated 90 degrees or at other orientations, and the spatially relative descriptors used herein should be interpreted accordingly.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the following description, numerous specific details are set forth in order to provide a thorough understanding of the present disclosure. The present disclosure may be practiced without some or all of these specific details. In other instances, well-known process structures and/or processes have not been described in detail in order not to unnecessarily obscure the present disclosure.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present disclosure refers to “one or more embodiments of the present disclosure.”

Hereinafter, embodiments of the present disclosure will be described in details with reference to attached drawings.

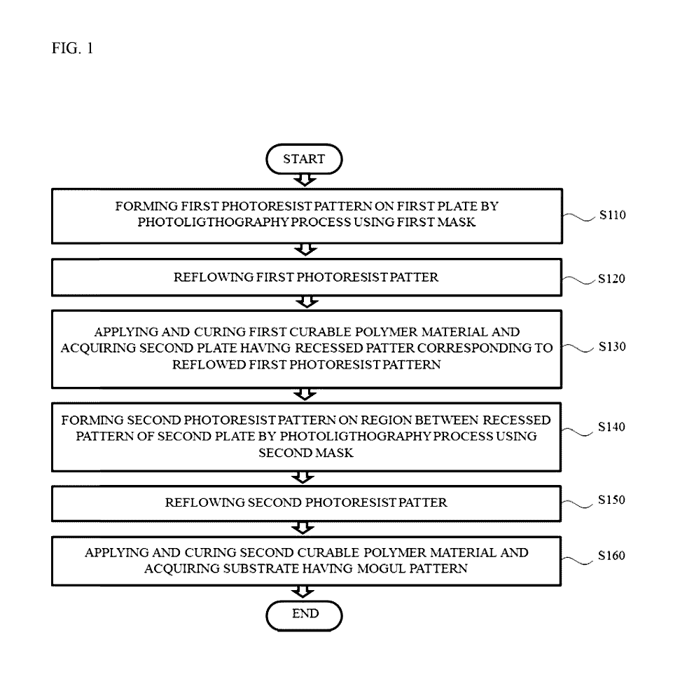

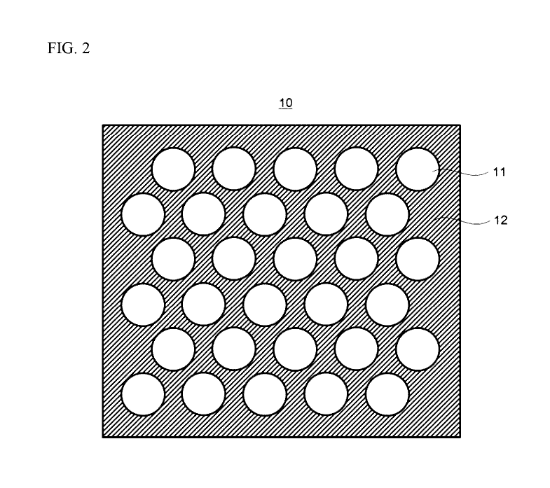

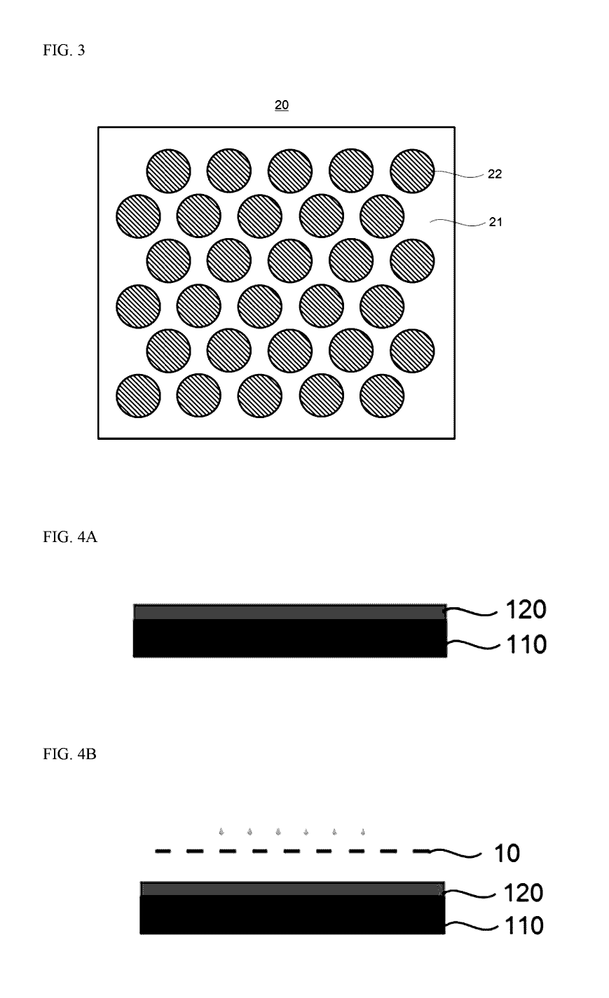

FIG. 1 shows a flow chart of a method for manufacturing a substrate for a stretchable device in accordance with one embodiment of the present disclosure. FIG. 2 and FIG. 3 shows top views of first and second masks as employed in the process in FIG. 1 respectively. FIG. 4A to FIG. 4K shows cross-sectional views of stages of the manufacturing process for the substrate for the stretchable device in FIG. 1.

As used herein, a term ‘mogul pattern’ may refer to a pattern having a plurality of depressions depressed from a virtual reference plane, and a continuous ridge formed between the depressions, wherein the ridge surrounds the depressions, and the depressions are regularly arranged and have substantially the same size and shape, wherein a combination of the depressions and the ridge has a continuous curved surface. Furthermore, as used herein, a term ‘mogul pattern’ may refer to a pattern having a plurality of bumps protruding from a virtual reference plane, and a continuous valley formed between the bumps, wherein the valley surrounds the bumps, and the bumps are regularly or irregularly arranged and have substantially the same size and shape, wherein a combination of the bumps and the valley has a continuous curved surface.

In one embodiment, mogul patterns with different sizes may be formed on a single substrate.

In one embodiment, a cross-section of the mogul pattern perpendicular to the virtual reference plane has peaks and valleys, wherein the peaks and valleys are repeatedly alternated and a combination of the peaks and valleys has a continuous curved line.

In one embodiment, the continuous curved face may periodically wavy. The period of the wave may or not be constant along the cross-section of the mogul pattern.

As used herein, a term ‘reverse mogul pattern’ may refer to a pattern shape-reverse to the mogul pattern.

Referring to FIG. 1 to FIG. 4, a method for manufacturing a substrate 100 for a stretchable electronic device in accordance with one embodiment of the present disclosure forming a first photoresist pattern 120a on a first plate 110 by a photolithography process using a first mask 10 as shown in FIG. 2 (S110); reflow the first photoresist pattern 120a (S120); applying and curing a first curable polymer material 130′ on the reflowed first photoresist pattern 120b and an exposed face of the first plate 110 and then obtaining a second plate 130 having a first reverse pattern formed therein, the first reverse pattern being shape-reverse to the reflowed first photoresist pattern 120b (S130); forming a second photoresist pattern 150a on top planar faces of protrusions of the first reverse pattern by a photolithography process using a second mask 20 as shown in FIG. 3 on the first reverse pattern (S140); reflow the second photoresist pattern 150a (S150); and applying and curing a second curable polymer material 100′ on the second photoresist pattern 150a and an exposed face of the second plate 130 and then forming a substrate 100 having a mogul pattern formed therein (S160).

As shown in FIG. 4A to FIG. 4C, in the operation S110, the first plate 110 may have a flat surface on which the first photoresist film 120 may be formed. The first plate 110 120 may not be limited specifically in terms of a material and structure as long as the first photoresist film 120 is formed thereon. For example, the first plate 110 may be made of a silicon wafer.

The first photoresist film 120 may be made of a positive type photoresist where a light-exposed portion thereof is developed on the first plate 110. In one embodiment, the first photoresist film 120 may have a thickness of about 10 to 40 μm. In one embodiment, the first photoresist film 120 may be formed by applying and thermally-treating the photoresist material on the first plate. For example, the first photoresist film 120 may be formed by applying the photoresist material on the silicon wafer using a spin coating, and performing a primary thermal treatment thereto at about 70 to 90° C. and performing a secondary thermal treatment thereto at about 110 to 130° C.

For patterning the first photoresist film 120, the first mask 10 as shown in FIG. 2 may be used which may have a plurality of first light-transmitting regions 11 spacedly arranged regularly, each having a circular or polygonal shape, and a first light-blocking region 12 formed between the first light-transmitting regions 11 to surround the light-transmitting regions 11. In one embodiment, each of the plurality of first light-transmitting regions 11 may have a circular shape, as shown in FIG. 2, for example, with a diameter of about 10 to 100 μm, and centers of the three neighboring first light-transmitting regions 11 may correspond to apexes of an equilateral or isosceles triangle respectively.

When the first photoresist film 120 is patterned by exposure and development using the first mask 10, pillar shaped openings may be formed in the first photoresist film 120, which may expose the first plate 110 and may have a top-view cross-sectional shape corresponding to the shape of the first light-transmitting region 11 in the first mask 10. Hereinafter, for the sake of convenience of illustration, the first photoresist film 120 having the pillar shaped openings formed therein having a top-view cross-sectional shape corresponding to the shape of the first light-transmitting region 11 will be referred to as a first photoresist pattern 120a.

In one embodiment, when each of first light-transmitting regions 11 of the first mask 10 has a circular shape, the first photoresist pattern 120a may have circular shaped openings exposing the first plate 110.

As shown in FIG. 4D, the operation S120 may include slowly heating the first photoresist pattern 120a to a first temperature above a melting point thereof, and, then, keeping the first photoresist pattern 120a at the first temperature for a predetermined time, and, then, slowly cooling the first photoresist pattern 120a to a room temperature. This reflow process may allow top angled corners of the first photoresist pattern 120a to be rounded, thus, allow the reflowed first photoresist pattern 120b to have a continuous curved top face.

As shown in FIG. 4E and FIG. 4F, the operation S130 may include applying the first curable polymer material 130′ on the exposed face of the first plate 110 and into the openings in the reflowed first photoresist pattern to form a constant layer thickness thereof, and curing the first curable polymer material 130′. In this connection, the first curable polymer material 130′ may be cured using an ultra-violet ray or thermal energy. In one embodiment, the first curable polymer material 130′ may be made of an organic/inorganic hybrid polymer material which may allow a micro pattern and have a good durability and be transparent. For example, the first curable polymer material 130′ may be made of a commercially available OrmoStamp® material.

Subsequently, the cured first curable polymer material 130′ may be separated from the first plate 110 to obtain a second plate 130 having the first reverse pattern formed thereon. Since the openings of the reflowed first photoresist pattern 120b expose a flat face of the first plate 110, the first reverse pattern may have protrusions corresponding to the openings respectively, each protrusion having a top flat face corresponding to the flat face of the first plate 110.

Further, in order to facilitate the separation between the first plate 110 and the cured first curable polymer material 130′, the reflowed first photoresist pattern 120b may be subjected to anti-adhesion treatment to the first curable polymer material 130′. For example, when the first curable polymer material 130′ is made of the OrmoStamp® material, for the anti-adhesion treatment, the first plate 110 having the reflowed first photoresist pattern 120b formed thereon and Trichloro(1H,1H,2H,2H-perfluorooctyl)-silane solution may be disposed in a vacuum chamber, and, then, the Trichloro(1H,1H,2H,2H-perfluorooctyl)-silane solution may be evaporated to form a release film on a surface of the reflowed first photoresist pattern 120b.

Furthermore, after the first curable polymer material 130′ is applied onto the reflowed first photoresist pattern 120b and the exposed face of the first plate 110, a glass substrate 140 with improved adhesion to the first curable polymer material 130′ may be pressed to the first curable polymer material 130′ and then the first curable polymer material 130′ may be cured. In order to acquire the glass substrate 140 with the improved adhesion to the first curable polymer material 130′, the glass substrate 140 may be subjected to a plasma treatment and then to a surface treatment using 3-aminopropyl-triethoxysilane solution.

As shown in FIG. 4G to FIG. 4F, the operation S140 may include applying a second photoresist film 150 on the second plate 130 having the first reverse pattern formed therein, and, then, patterning the second photoresist film 150 using the second mask 20.

The second photoresist film 150 may be made of a positive type photoresist material where an exposed portion thereof is developed. In one embodiment, when, in the first mask 10, a width of the first light-blocking region 12 between the neighboring first light-transmitting regions 11 is smaller than a diameter of the first light-transmitting region 11, the second photoresist film 150 may have a thickness smaller than that of the first photoresist film 120. For example, the second photoresist film 150 may have a thickness of about 20 to 50 μm.

For patterning the second photoresist film 150, the second mask 20 as shown in FIG. 3 may be employed which may have a plurality of second light-blocking regions 22 corresponding to the first light-transmitting regions 11 of the first mask 10 respectively, for example, in terms of a shape, size and arrangement, and a second light-transmitting region 21 corresponding to the first light-blocking region 12 of the first mask 10, for example, in terms of a shape, size and arrangement.

When the second photoresist film 150 is patterned using the second mask 20, as shown in FIG. 4H, a portion of the second photoresist film 150 corresponding to a valley region of the first reverse pattern is exposed and developed, and, thus, only a portion of the second photoresist film 150 corresponding to the protrusion of the first reverse pattern remains.

As shown in FIG. 4I, the operation S150 may include slowly heating the second photoresist pattern 150a to a second temperature above a melting point thereof, keeping the second photoresist pattern 150a at the second temperature for a predetermined time, and, subsequently, slowly cooling the second photoresist pattern 150a to a room temperature. This reflow process may allow top angled corners of the second photoresist pattern 150a to be rounded and allow a discontinuity in a curve between the second photoresist pattern 150a and the first reverse pattern to have a continuity in a curve. In this way, the combination of the reflowed second photoresist pattern 150b and the first reverse pattern may have a continuity in a curve.

As shown in FIG. 4J and FIG. 4K, the operation S160 may include applying and curing a second curable polymer material 100′ on the reflowed second photoresist pattern 150b and the first reverse pattern formed on the second plate 130, and separating the cured second curable polymer material 100′ from the second plate, thereby to acquire the substrate 100 having a mogul pattern formed thereon. In this connection, the second curable polymer material 100′ may fill recess regions defined by the reflowed second photoresist pattern 150b and the first reverse pattern so as to form a layer with a predetermined thickness. That is, the second curable polymer material 100′ may include a first portion filling the recesses and a second portion deposited on the first portion.

In one example, the second curable polymer material 100′ may be made of PDMS (polydimethylsiloxane).

Further, in order to facilitate the separation between the substrate 100 having the mogul pattern formed therein and the second plate 130, the second plate 130 may be subjected to an anti-adhesion treatment prior to the application of the second curable polymer material 100′ thereto. For example, when the second curable polymer material 100′ is made of PDMS, a release film may be formed on the second plate 130 via evaporation of TMCS (Chlorotrimethylsilane) solution.

Further, in order to simplify a manufacturing process of the substrate for a stretchable device and enable the mass production thereof, the method for manufacturing the substrate for a stretchable device 100 in accordance with one embodiment of the present disclosure may further include applying and curing a third curable polymer material on the mogul pattern of the substrate 100 and then removing the third curable polymer material from the substrate 100, to form a master mold having a reverse mogul pattern formed thereon. Using the master mold, a substrate having the mogul pattern formed therein may be acquired. The third curable polymer material may be made of a polymer material with good durability and thermal stability. For example, the third curable polymer material may be made of polyurethane acrylate (PUA).

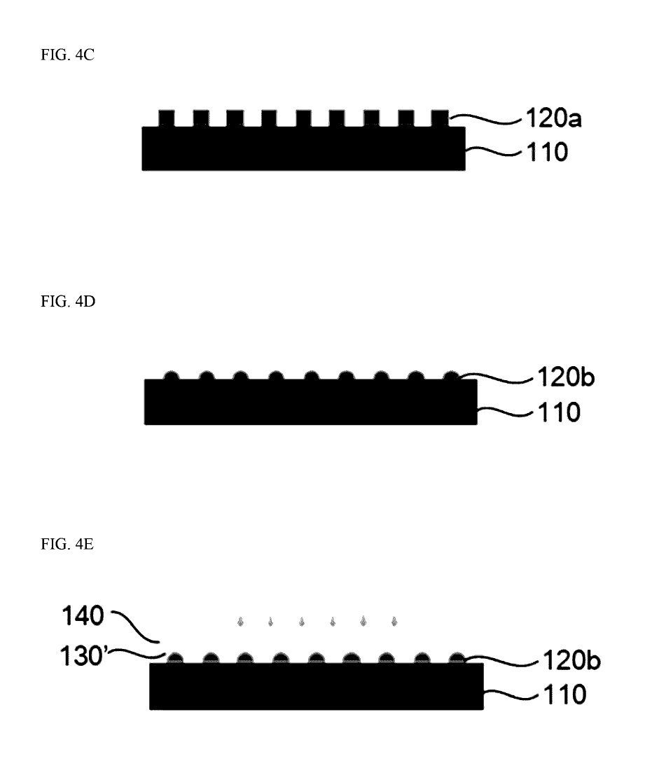

FIG. 5A shows an image of a substrate for a stretchable device in accordance with one embodiment of the present disclosure. FIG. 5B shows a cross-sectional view taken a line A-A′ in FIG. 5A.

Referring to FIG. 5A and FIG. 5B, the substrate for a stretchable device in accordance with one embodiment of the present disclosure, as produced by the above-defined method may have a mogul pattern formed therein.

In one embodiment, as for the mogul pattern, a ratio between a pitch W between neighboring peaks and a height H between the peak portion and the valley portion may be in a range of about 1:0.5 to 1:1.5. When a ratio H/W of the height H to the pitch W is below 0.5, a stretchability of an electrode structure or electronic device formed on the substrate may be below 20%. When a ratio H/W of the height H to the pitch W is above 1.5, the electrode structure or electronic device may not be formed on the substrate due to a large waviness of a surface of the substrate. In one example, when a ratio H/W of the height H to the pitch W is about 0.5 to 1.5, the expected stretchability of an electrode structure or electronic device formed on the substrate may reach about 20 to 80%.

The substrate for a stretchable device in accordance with one embodiment of the present disclosure may be used as a substrate for various electronic devices. In this connection, on the mogul pattern, there may be formed thin-film structure including a conductive thin-film, an insulating film, a semiconductor thin-film, etc. As noted above, the substrate for the stretchable device in accordance with one embodiment of the present disclosure may have the mogul pattern as formed using the above-defined method, wherein the mogul pattern may not have angled corner portions, that is, may be continuous in a curve. Thus, when the substrate 100 for the stretchable device 100 is stretched, the thin-film structure formed on the mogul pattern may have a minimum deformation and may be free of the thin-film structure damage due to the stress concentration at the angled corner portions, that is, the discontinuity in a curve.

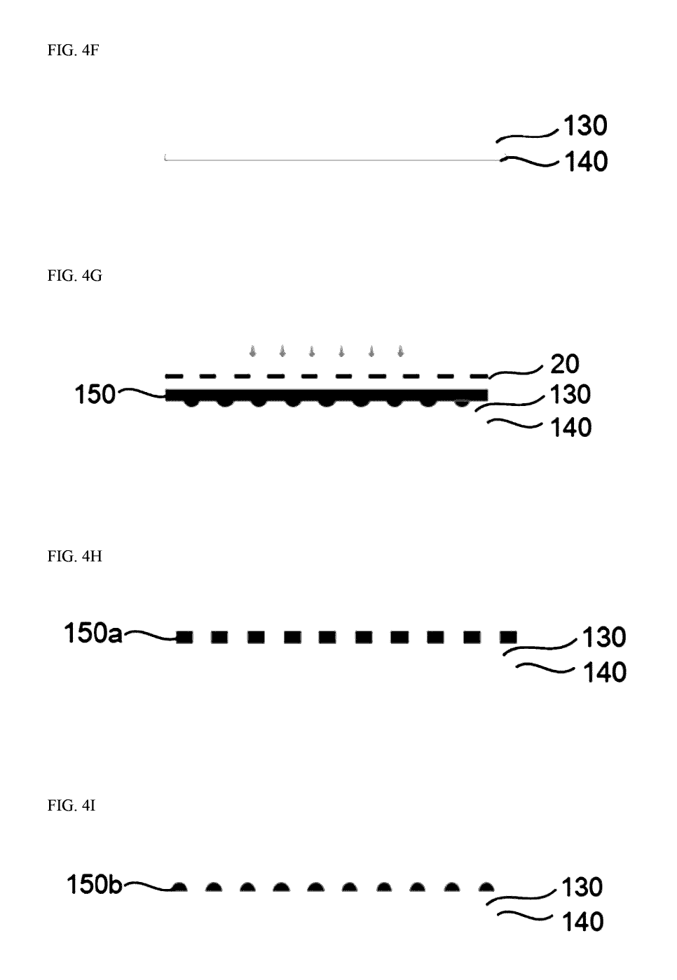

Hereafter, with reference to FIG. 6 to FIG. 8, tensile characteristics of a substrate for a stretchable device in accordance with one embodiment of the present disclosure will be described.

FIG. 6 shows a graph indicating resistance variations of gold thin-film patterns based on tensile strains of two substrates, wherein one substrate is a conventional flat substrate and the other is a substrate for a stretchable device, having a mogul pattern formed thereon, in accordance with one embodiment of the present disclosure, wherein the gold thin-film patterns are formed on the two substrates respectively, wherein each of the gold thin-film patterns has an initial length 1 cm and thickness 70 nm.

Referring to FIG. 6, when the gold thin-film pattern is formed on the conventional flat substrate, the resistance of the gold thin-film pattern may rapidly increase based on the tensile strain of the substrate. For example, when the tensile strain of the substrate reaches 5%, a ratio of a resistance variation ΔR to an initial resistance R₀ of the gold thin-film pattern, that is, a ratio ΔR/R₀ reaches above 6.5. To the contrary, when the gold thin-film pattern is formed on the present substrate, the resistance of the gold thin-film pattern may not rapidly increase based on the tensile strain of the substrate. For example, when the tensile strain of the substrate reaches 50%, a ratio of a resistance variation ΔR to an initial resistance R₀ of the gold thin-film pattern, that is, a ratio ΔR/R₀ reaches below 5.6. In particular, when the gold thin-film pattern is formed on the present substrate with the mogul pattern, the resistance of the gold thin-film pattern may slightly increase in a region in which the tensile strain of the substrate is below 10%. Specifically, when the tensile strain of the substrate is below 10%, a ratio ΔR/R₀ of the gold thin-film pattern may reach only 0.4.

Further, the present applicants have checked that, when the gold thin-film pattern is formed on the present substrate with the mogul pattern and the substrate is repeatedly tensile stretched with a strain of 50%, a crack may not occur in the gold thin-film pattern during 1000 times repetition of tensile stretching.

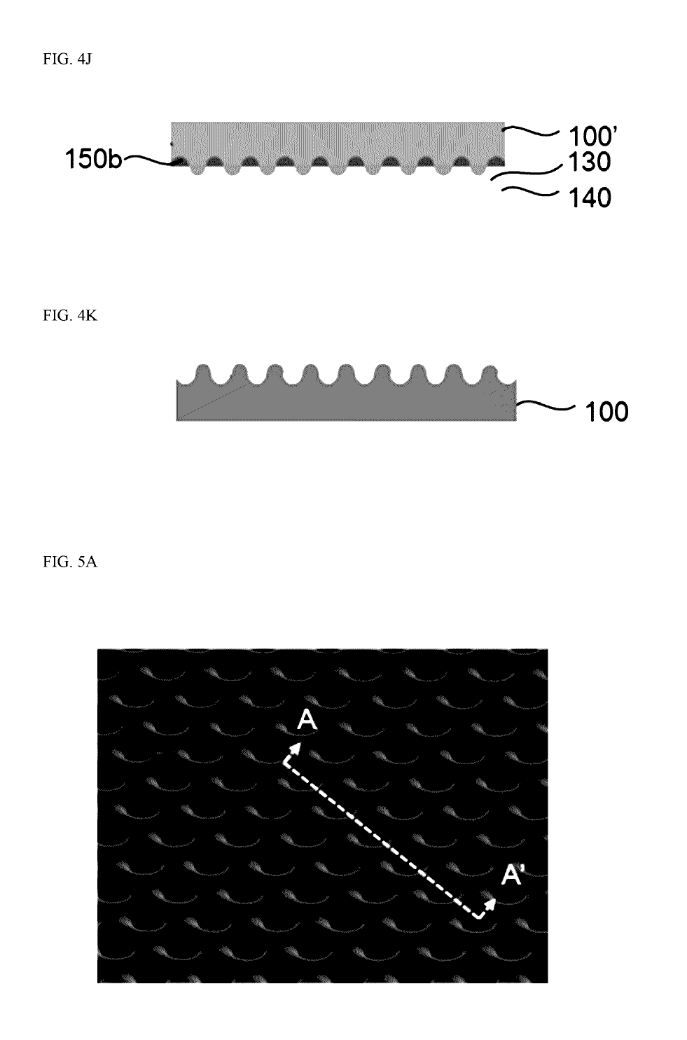

FIG. 7 shows a graph indicating resistance variations of PEDOT:PSS thin-film patterns based on tensile strains of two substrates, wherein one substrate is a conventional flat substrate and the other is a substrate for a stretchable device, having a mogul pattern formed thereon, in accordance with one embodiment of the present disclosure, wherein the PEDOT:PSS thin-film patterns are formed on the two substrates respectively, wherein each of the PEDOT:PSS thin-film patterns has an initial length 1 cm and thickness 160 nm.

Referring to FIG. 7, when the PEDOT:PSS thin-film pattern is formed on the conventional flat substrate, the resistance of the PEDOT:PSS thin-film pattern may slightly increase in a region in which the tensile strain of the substrate reaches 5%, but may rapidly increase in a region in which the tensile strain of the substrate exceeds 5%. To the contrary, as for the PEDOT:PSS thin-film formed on the present substrate, when the tensile strain of the substrate reaches 50%, a ratio of a resistance variation ΔR to an initial resistance R₀ of the PEDOT:PSS thin-film pattern, that is, a ratio ΔR/R₀ is below 4.5. In particular, when the PEDOT:PSS thin-film pattern is formed on the present substrate with the mogul pattern, the resistance of the PEDOT:PSS thin-film pattern may slightly increase in a region in which the tensile strain of the substrate is below 15%. Specifically, when the tensile strain of the substrate is 15%, a ratio ΔR/R₀ of the PEDOT:PSS thin-film pattern may reach only 0.45.

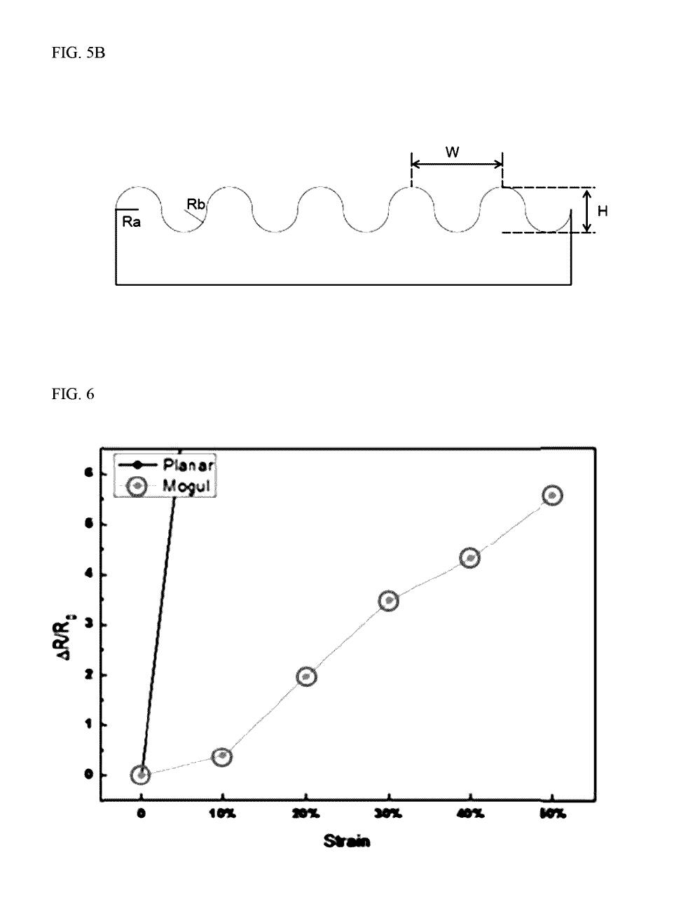

FIG. 8 shows a graph indicating resistance variations of ITO thin-film patterns based on tensile strains of two substrates, wherein one substrate is a conventional flat substrate and the other is a substrate for a stretchable device, having a mogul pattern formed thereon, in accordance with one embodiment of the present disclosure, wherein the ITO thin-film patterns are formed on the two substrates respectively, wherein each of the ITO thin-film patterns has an initial length 1 cm and thickness 160 nm.

Referring to FIG. 8, when the ITO thin-film pattern is formed on the conventional flat substrate, the resistance of the ITO thin-film pattern may slightly increase in a region in which the tensile strain of the substrate reaches 5%, but may rapidly increase in a region in which the tensile strain of the substrate exceeds 5%. To the contrary, as for the ITO thin-film formed on the present substrate, when the tensile strain of the substrate reaches 15%, a ratio of a resistance variation ΔR to an initial resistance R₀ of the ITO thin-film pattern, that is, a ratio ΔR/R₀ is below 4.5.

Based on the above experiment results, it may be confirmed that, when a thin-film structure made of a non-stretchable material is formed on the substrate for a stretchable device, having the mogul pattern formed thereon, in accordance with one embodiment of the present disclosure, the substrate may realize a minimization of a deformation of the thin-film structure made of the non-stretchable material and a spreading of the stress thereto, if it occurs, thereby to prevent the damage of the thin-film structure.

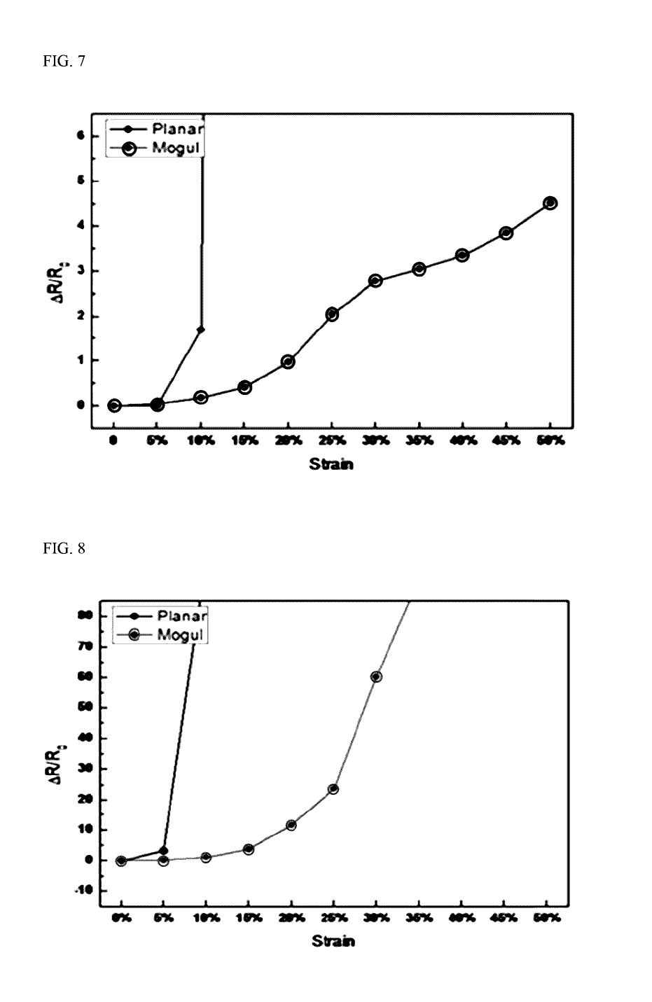

FIG. 9 shows a perspective view of a gas sensing device in accordance with one embodiment of the present disclosure.

Referring to FIG. 9, the gas sensing device 1000 in accordance with one embodiment of the present disclosure may include a substrate 1100 having a mogul pattern formed on a first face thereof; first and second electrodes 1200A, 1200B formed on the mogul pattern, the first and second electrodes 1200A, 1200B being spaced from each other, and a sensing material 1300 formed on the mogul pattern to be electrical contact with the first electrode 1200A and the second electrode 1200B.

The substrate 1100 may refer to the substrate with the mogul pattern, as described above, in accordance with one embodiment of the present disclosure.

The first and second electrodes 1200A, 1200B may be formed on the mogul pattern. The first and second electrodes 1200A, 1200B may be spaced from each other. Each of the first and second electrodes 1200A, 1200B may be made of a metal, conducive polymer, conductive oxide, etc.

The sensing material 1300 may be made of a material reacting with a certain gas to exhibit a change in an electrical property. The sensing material 1300 may be formed on the mogul pattern. The sensing material 1300 may electrically contact the first and second electrodes 1200A, 1200B. In one embodiment, when the gas sensing device 1000 senses NO₂, the sensing material 1300 may be made of a reduced graphene oxide. When the NO₂ is absorbed to the reduced graphene oxide, electrons may migrate from the reduced graphene oxide to the NO₂, to increase a hole concentration in the reduced graphene oxide. Thus, the reduced graphene oxide may have a changed electrical conductance. The gas sensing device 1000 in accordance with one embodiment of the present disclosure may sense NO₂ by measuring a variation in the electrical conductance of the sensing material 1300 via the first and second electrode 1200A, 1200B.

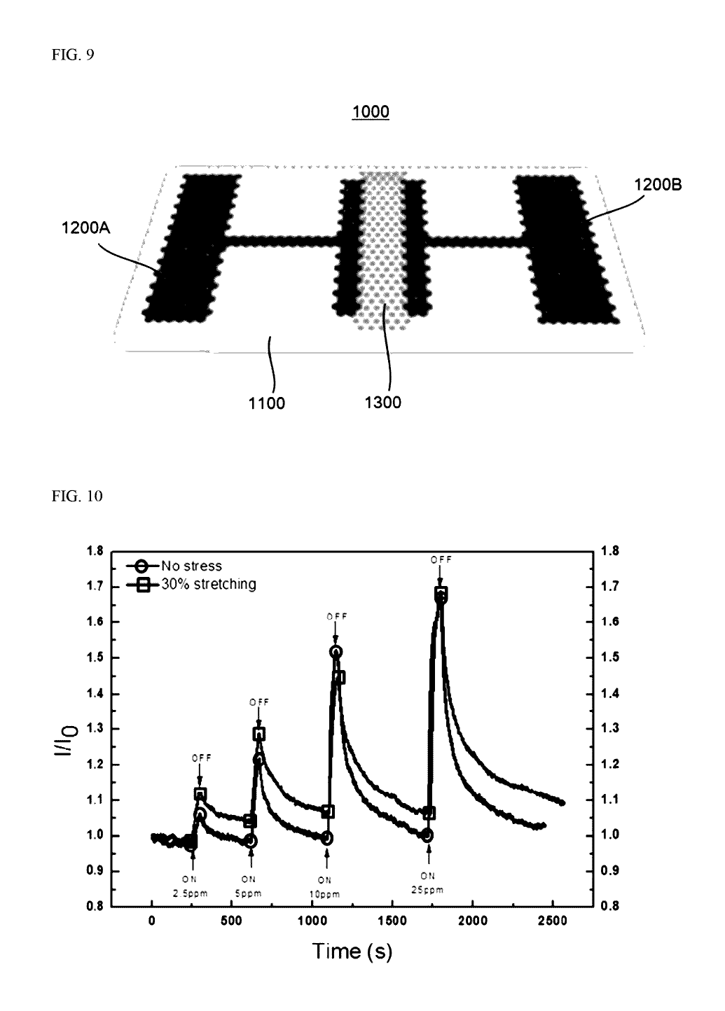

FIG. 10 shows a graph indicating a variation in the electrical conductance of the gas sensing device based on NO₂ concentrations. As for the gas sensing device, the sensing material 1300 may be made of a reduced graphene oxide, and each of the first and second electrodes 1200A, 1200B may be made of gold (Au). In FIG. 10, a black curve indicates measurements when the substrate is not deformed, while a blue curve indicates measurements when the substrate has deformed by 30%.

Referring to FIG. 10, it may be confirmed that the current variations when the substrate is not deformed has the identical change trend with the current variations when the substrate has deformed by 30%. From this, the NO₂ sensing device in accordance with one embodiment of the present disclosure may reliably sense NO₂ even when the substrate has deformed by 30%.

The above description is not to be taken in a limiting sense, but is made merely for the purpose of describing the general principles of exemplary embodiments, and many additional embodiments of this disclosure are possible. It is understood that no limitation of the scope of the disclosure is thereby intended. The scope of the disclosure should be determined with reference to the Claims. Reference throughout this specification to “one embodiment,” “an embodiment,” or similar language means that a particular feature, structure, or characteristic that is described in connection with the embodiment is included in at least one embodiment of the present disclosure. Thus, appearances of the phrases “in one embodiment,” “in an embodiment,” and similar language throughout this specification may, but do not necessarily, all refer to the same embodiment.

What is claimed is:

1. A substrate for a stretchable device, the substrate having a mogul pattern formed thereon, wherein the mogul pattern has a plurality of depressions depressed from a virtual reference plane, and a continuous ridge formed between the depressions, wherein the ridge surrounds the depressions, and the depressions are regularly arranged and have substantially the same size and shape, wherein a combination of the depressions and the ridge has a continuous curved surface.

2. The substrate of claim 1, wherein a cross-section of the mogul pattern perpendicular to the virtual reference plane has peaks and valleys, wherein the peaks and valleys are repeatedly alternated and a combination of the peaks and valleys has a continuous curved line.

3. The substrate of claim 2, wherein a ratio between a pitch between neighboring peaks and a height from each valley to each peak is in a range of about 1:0.5 to 1:1.5.

4. A method for manufacturing a substrate for a stretchable device, the method comprising:
(a) forming a first photoresist pattern on a first plate by a photolithography process using a first mask, wherein the first mask has a plurality of first light-transmitting regions spacedly arranged regularly and a first light-blocking region surrounding the first light-transmitting regions, wherein each light-transmitting region has a circular or polygonal shape;
(b) reflowing the first photoresist pattern;
(c) applying and curing a first curable polymer material on an exposed face of the first plate and on the reflowed first photoresist pattern, and acquiring a second plate having a reverse pattern formed thereon, wherein the reverse pattern is shape-reverse to the reflowed first photoresist pattern, wherein the reverse pattern is made of the first curable polymer material;
(d) forming a second photoresist pattern on the reverse pattern by a photolithography process using a second mask, wherein a non-removed portion of the second photoresist pattern overlaps a protrusion of the reverse pattern, and a removed portion of the second photoresist pattern overlaps a non-protrusion of the reverse pattern, wherein the second mask has second light-blocking regions corresponding to the first light-transmitting regions respectively, and a second light-transmitting region corresponding to the first light-blocking region;
(e) reflowing the second photoresist pattern; and
(f) applying and curing a second curable polymer material on the reflowed second photoresist pattern and a non-protrusion of the reverse pattern, and acquiring the substrate having a mogul pattern formed therein, wherein the mogul pattern is shape-reverse to a combination of the reflowed second photoresist pattern and the non-protrusion of the reverse pattern, wherein the mogul pattern is made of the second curable polymer material.

5. The method of claim 4, wherein the operation (a) includes forming a first photoresist film on the first plate and patterning the first photoresist film using the first mask, wherein the first photoresist film has a thickness of about 10 to 40 μm,
wherein the operation (b) includes forming a second photoresist film on the reverse pattern and patterning the second photoresist film using the second mask, wherein the second photoresist film has a thickness of about 20 to 50 μm.

6. The method of claim 4, wherein each of the first light-transmitting regions in the first mask has a circular shape with a diameter of about 10 to 100 μm.

7. The method of claim 4, wherein the combination of the reflowed second photoresist pattern and the non-protrusion of the reverse pattern has a continuous curved surface, and, thus, the mogul pattern has a continuous curved surface.

8. The method of claim 4, wherein the operation (c) includes:
forming a release film on the reflowed first photoresist pattern;
applying the first curable polymer material on the release film;
pressuring the first curable polymer material using a glass substrate with improved adhesion to the first curable polymer material;
curing the first curable polymer material; and
removing the cured first curable polymer material from the reflowed first photoresist pattern using the release film.

9. The method of claim 4, further comprising:
applying and curing a third curable polymer material on the mogul pattern and acquiring a master mold having a reverse mogul pattern formed thereon, wherein the reverse mogul pattern is made of the third curable polymer material, and the reverse mogul pattern is shape-reverse to the mogul pattern; and
applying and curing the second curable polymer material on the reverse mogul pattern and acquiring the substrate having the mogul pattern formed thereon, wherein the mogul pattern is made of the second curable polymer material.

10. The method of claim 9, wherein the third curable polymer material includes polyurethane acrylate.

11. An electronic device comprising:
a substrate having a mogul pattern formed thereon, wherein the mogul pattern has a plurality of bumps protruding from a virtual reference plane, and a continuous valley formed between the bumps, wherein the valley surrounds the bumps, and the bumps are regularly or irregularly arranged and have substantially the same size and shape, wherein a combination of the bumps and the valley has a continuous curved surface; and
an electrical conductive thin-film structure formed on the mogul pattern.

12. The device of claim 11, wherein the electrical conductive thin-film structure is made of a metal, a conducive polymer, and/or a conductive oxide.

13. The device of claim 11, wherein the electronic device is a sensing device, wherein the electrical conductive thin-film structure includes first electrode and second electrodes spaced from each other, wherein the electronic device is disposed on the mogul pattern, and wherein the electronic device further comprises a sensing material to sense a target material, wherein the sensing material electrically contacts the first electrode and the second electrode.

14. The device of claim 13, wherein the sensing material includes a reduced graphene oxide.
